(19) United States
(12) Patent Application Publication
(10) Pub. No.: US 2017/0049405 A1
(43) Pub. Date: Feb. 23, 2017

(54) MEDICAL ALERT SYSTEM

(71) Applicant: Pamela Jordan, Delhi, LA (US)

(72) Inventor: Pamela Jordan, Delhi, LA (US)

(21) Appl. No.: 14/832,119

(22) Filed: Aug. 21, 2015

(51) Int. Cl. A61B 5/00; G08B 21/02

(52) U.S. Cl. CPC A61B 5/746; G08B 21/02; A61B 5/0022

(57) ABSTRACT

A system for transmitting medical coding alerts including: a database of users; a central communication processor, with a central communication processor controls alerts to users; a plurality of patient alerts, where the processor transmits a patient alert to the end users; software, where the software provides instructions for the communications processor that controls the transmission of patient alerts to users; and user software, where the user software is distributed to each user via a smart phone. Each user is an authorized medical professional and the patient alerts relate to immediate patient conditions. The patient alerts include common medical codes, including code blue. The user software includes a user icon displayed on the user's smart phone providing the alert.

### BACKGROUND OF THE INVENTION

Field of Invention

The present invention relates to a medical alert system for the transmission of medical alert codes.

Description of Related Art

Medical professionals use audio emergency codes in hospitals to indicate conditions of various patients at various times. The code system enables the communication of patient status and/or emergency conditions with a minimum amount of specific information being transmitted. Many times these codes are announced over a PA system so that hospital personnel are clearly notified of an urgent emergency. Typical codes may include one common code, Code Blue indicating a cardiac respiratory emergency. Further codes may include Code Pink, White, Pediatric and certain other numbers are used for codes. One drawback to the public address coding system of a medical center is that individuals may wander outside the PA system range for various reasons. As a result, it would be advantageous to have a similar system set up that could be implemented through a mobile application for use on a smart phone. Many individuals use various types of smart phones as part of their personal communications systems. As a result, if such an application is developed for medical personnel to receive code alerts through the application for an indication of a patient's status.

### SUMMARY OF THE INVENTION

The present invention relates a system for transmitting medical coding alerts including: a database of users; a central communication processor, with a central communication processor controls alerts to users; a plurality of patient alerts, where the processor transmits a patient alert to the end users; software, where the software provides instructions for the communications processor that controls the transmission of patient alerts to users; and user software, where said user software is distributed to each user via a smart phone. Each user is an authorized medical professional and the patient alerts relate to immediate patient conditions. The patient alerts include common medical codes, including code blue. The user software includes a user icon displayed on the user's smart phone providing the alert.

### BRIEF DESCRIPTION OF DRAWINGS

FIG. 1 depicts a series of application icons used in conjunction a smart phone with a coding application according to the present invention implemented.

### DETAILED DESCRIPTION

The present invention relates to a mobile system and application for implementation onto a smart phone. This coding system provides a visible icon on a smart phone that implements the coding system of a hospital or health care facility. The coding system as a result provides alerts to medical personnel of a patient's status as needed. This coding system is conveniently available for hospital personnel who carry a smart phone. Smart phones are very prevalent throughout society and as a result such implementation could be very easily transitioned for medical personnel.

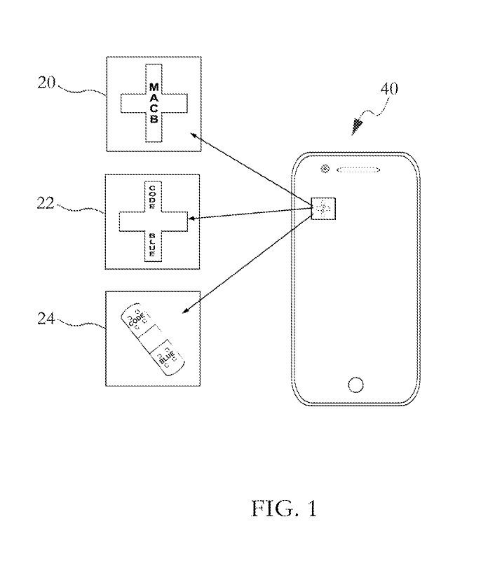

In reference to FIG. 1, a series of icons depicting a medical alert coding system is shown. As is well known Code Blue is a common alertcode used in the medical hospitals. Various other types of codes may be implemented based upon the health care facility and the coding system implemented in a particular facility. FIG. 1 depicts a series of these medical coding icons for use with the present medical coding application. A first application icon 20 is shown in FIG. 1 along with second and third embodiments of the coding icon. The Icon 20 depicts a cross with initials used through the center of the cross indicating an applicable code transmission. Icon 22 also depicts a cross where the code is spelled out through the center of the cross giving a clear indication of the code being transmitted. As a third and alternative embodiment, Icon 24 depicts a Band Aid with coding text provided within the Band Aid.

This particular system is implemented through software that is downloaded onto a Smart Phone 40 as shown in FIG. 1. This software is controlled by a central communication system within the medical center. This central communication center includes a plurality of users that gain access to the medical coding application. As stated this application may be supplied over various application distribution systems provided for existing smart phones. A user may be given exclusive access to the medical coding application according to the present invention. Once the user gains access a designated icon is available on their respective smart phone as shown in FIG. 1. Once this application is implemented a control system is provided to transmit the coding to all users within the system. Access and deactivation of access is provided through a central control system that controls the users and users' access to the medical alerting application. The instant invention has been shown and described in what it considers to be the most practical and preferred embodiments. It is recognized, however, that departures may be made there from within the scope of the invention and that obvious modifications will occur to a person skilled in the art.

1. A system for transmitting medical coding alerts comprising:
a database of users;
a central communication processor, with a central communication processor controls alerts to users;
a plurality of patient alerts, where the processor transmits a patient alert to the end users, where the patient alerts relate to immediate patient conditions;
software, where the software provides instructions for the communications processor that controls the transmission of patient alerts to users; and
user software, where said user software is distributed to each user via a smart phone.

2. The system for transmitting medical coding alerts according to claim 1, where each user is an authorized medical personnel.

3. (canceled)

4. The system for transmitting medical coding alerts according to claim 1, where patient alerts include common medical codes, including code blue.

5. The system for transmitting medical coding alerts according to claim 1, where the user software includes a user icon displayed on the user's smart phone.
